(19) United States
(12) Patent Application Publication
(10) Pub. No.: US 2023/0165876 A1
(43) Pub. Date: Jun. 1, 2023

(54) METHYLTHIONINIUM COMPOUNDS FOR USE IN THE TREATMENT OF COVID-19

(71) Applicant: WisTa Laboratories Ltd., Singapore (SG)

(72) Inventors: Claude Michel Wischik, Aberdeen, Aberdeens (GB); Mohammad Arastoo, Aberdeen, Aberdeenshire (GB); Michael Philip Mazanetz, Newlands, Glasgow (GB)

(73) Assignee: WisTa Laboratories Ltd., Singapore (SG)

(21) Appl. No.: 17/922,837

(22) PCT Filed: Apr. 30, 2021
(86) PCT No.: PCT/EP2021/061481

§ 371 Date: Nov. 2, 2022

(30) Foreign Application Priority Data
May 5, 2020 (GB) 2006659.3

(51) Int. Cl. A61K 31/5415; A61K 31/4706

(52) U.S. Cl. CPC A61K 31/5415; A61K 31/4706

(57) ABSTRACT

T he present invention provides methods of treating COV-ID-19 in a subject using methylthioninium compounds.

### TECHNICAL FIELD

The present invention relates generally to methods and materials for use in the treatment of COVID-19.

### BACKGROUND ART

The emergence of novel SARS coronavirus 2 (SARS-CoV-2) in 2019 has triggered an ongoing global pandemic of severe pneumonia-like disease designated as coronavirus disease 2019 (COVID-19). COVID-19 poses a major healthcare and economic threat globally.

Repositioning of known drugs can significantly accelerate the development and deployment of therapies for COVID-19 and therefore there is an interest in profiling known drugs which may inhibit viral replication. For example Riva et al. (“A Large-scale Drug Repositioning Survey for SARS-CoV-2 Antivirals.” bioRxiv (2020)) profiled approximately 12,000 clinical-stage or FDA approved small molecules and reported the identification of 30 known drugs that inhibited viral replication under the tested conditions, of which six were characterized for cellular dose-activity relationships, and showed effective concentrations which they believed to be likely to be commensurate with therapeutic doses in patients. These include the PIKfyve kinase inhibitor Apilimod, cysteine protease inhibitors MDL-28170, Z LVG CHN2, VBY-825, and ONO 5334, and the CCR1 antagonist MLN-3897.

However screening of this type focusses on only a single attribute of SARS-CoV-2 (here: viral replication in Vero E6 cells) and the concentration of compound used in the screen (here: 5 µM) may not be optimal for detecting all promising candidates, or predictive of appropriate in vivo therapeutic doses.

Furthermore COVID-19 has been reported to be particularly harmful in vulnerable patients such as the elderly. Many potential therapeutics may not be suitable for use in that patient group.

Thus it can be seen that providing compounds or combinations of compounds which can be used safely in an elderly population, can target multiple attributes of the COVID-19 aetiology, and providing dosage information applicable to that, provides a useful contribution to the art.

### DISCLOSURE OF THE INVENTION

The present invention provides for the use of certain hydromethylthionine salts (referred to as “LMTX” below) as a monotherapy or combination therapy (with chloroquine/hydroxychloroquine) for the treatments of COVID-19. In the light of the disclosure herein, it can be expected that such treatment can provide a number of beneficial or synergistic treatment effects.

As explained hereinafter preliminary unconfirmed research suggests that MTC (methylthioninium chloride, methylene blue) may have the ability to lower the incidence of vulnerable patients reporting symptoms consistent with COVID-19 (Henry et al., 2020).

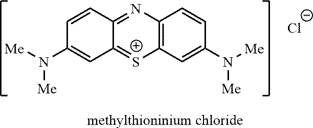

LMTX delivers the same MT (methylthionine) moiety systemically, but is more suitable for oral and intravenous use than MTC as it has improved absorption, red cell penetration and deep compartment distribution (Baddeley et al., 2015). LMTX can be used at a substantially lower dose than MTC and is thus better tolerated.

Independently of MTC, the antimalarial compound chloroquine and the related hydroxychloroquine are currently being investigated globally to assess their effectiveness as antiviral drugs against SARS-CoV-2.

However, chloroquine has a narrow therapeutic ratio such that significant electrophysiological effects occur at plasma concentrations approaching the micromolar range which is required for pharmacological activity. A Brazilian trial of chloroquine diphosphate for COVID-19 cases at two doses (https://doi.org/10.1101/2020.04.07.20056424) was reportedly halted because of cardiac deaths.

LMTX has a more benign safety profile. The inventors have established that LMTX does not demonstrate cardiotoxicity.

The present specification discloses that not only can LMTX provide benefits to subjects in permitting reduction of viral load, but it can also complex heme which may, either directly or indirectly, provide supportive activity in COVID-19, and further more may mitigate damage to pulmonary endothelium resulting from inflammatory, hyperoxic and mechanical injury to lung. In combination with the lack of cardiotoxicity, that limits the dose and duration of treatment with chloroquine, LMTX can provide a safer approach to treatment either alone or in combination with that agent.

LMTX salts have previously been described in general terms for treatment of viral disease (see WO2007/110627, and WO2012/107706) but not for the treatment of COVID-19 or other coronaviruses.

Thus in one aspect there is disclosed a method of therapeutic treatment of COVID-19 in a subject,
which method comprises administering to said subject a methylthioninium (MT)-containing compound,
wherein the MT-containing compound is an LMTX compound of the following formula:

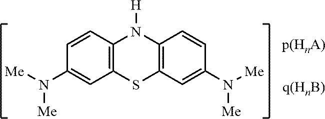

wherein each of HₙA and HₙB (where present) are protic acids which may be the same or different, and wherein p = 1 or 2; q = 0 or 1; n = 1 or 2; (p + q) x n = 2, or a hydrate or solvate thereof.

Preferably said administration provides a total daily oral dose of between 10 and 30 mg of MT to the subject per day, optionally split into 2 or more doses, or said administration provides a total daily intravenous (IV) dose of between 10 and 25 mg of MT to the subject per day.

In one embodiment the subject is a human who has been diagnosed as having COVID-19. The method may comprise making said diagnosis.

In one aspect there is disclosed a method of prophylactic treatment of COVID-19 in a subject,
which method comprises administering to said subject a methylthioninium (MT)-containing compound,
wherein the MT-containing compound is an LMTX compound as defined above, or a hydrate or solvate thereof.

Preferably said administration provides a total daily oral dose of between 10 and 30 mg of MT to the subject per day, optionally split into 2 or more doses, or said administration provides a total daily intravenous (IV) dose of between 10 and 25 mg of MT to the subject per day,

In one embodiment the subject is a human who has been assessed as having suspected or probable COVID-19 e.g. a subject who has been in close contact with one or more COVID-19 cases; a subject who is at least 65 years old; a subject living in a nursing home, care home, or long-term care facility; a subject with a relevant underlying medical condition.

As explained herein an appropriate oral dosage of MT which is appropriate to the combined aims of the invention is around 10 - 30 mg/MT day.

The total daily dose may be between 12 and 27 mg.

The total daily dose may be between 14 and 20 mg.

The total daily dose may be between 15 and 18 mg.

The total daily dose may be about 10, 11, 12, 13, 14, 15, 16, 17, 18, 19, 20, 21, 22, 23, 24, 25, 26, 27, 28, 29, or 30 mg.

In one embodiment the dose is 16 mg MT, which equates to about 27 mg LMTM. That is, as is the same as that required for optimal activity in AD.

The total daily dose of the compound may be administered as a split dose twice a day or three times a day.

As explained below, when administering the MT dose split in a larger number of doses/day it may be desired to use a smaller total amount within the recited range, compared to a single daily dosing, or a smaller number of doses per day.

For subjects needing respiratory support (or who otherwise may not be readily able to ingest the LMTX orally) it may be preferred to administer LMTX intravenously.

One daily IV dose is between 10 and 25 mg of MT to the subject per day.

The total daily IV dose may be about 10, 11, 12, 13, 14, 15, 16, 17, 18, 19, 20, 21, 22, 23, 24, 25 mg.

A preferred total daily IV dose is between 14 and 20 mg of MT to the subject per day.

Dosing may be by continuous infusion, or intermittent (e.g. 2, 4 or 6 times per day, for a few minutes each time).

For example, a smaller dose is preferred for continuous infusion (for example 0.6 mg/hr or around 14 mg/day) compared to intermittent dosing (4.8 mg administered over 5 minutes every 6 hours or around 20 mg/day).

Intermediate dosages for intermediate types of administration can be derived from these values in the light of the disclosure herein by those skilled in the art.

### LMTX Compounds

Preferably the LMT compound is an “LMTX” compound of the type described in WO2007/110627 or WO2012/107706.

Thus the compound may be selected from compounds of the following formula, or hydrates or solvates thereof:

| | |
|---|---|
| 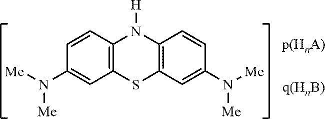 | Options: p=1,2 q= 0, 1 n = 1, 2 (p + q) x n = 2 |

Each of HₙA and HₙB (where present) are protic acids which may be the same or different.

By “protic acid” is meant a proton (H⁺) donor in aqueous solution. Within the protic acid A-or B- is therefore a conjugate base. Protic acids therefore have a pH of less than 7 in water (that is the concentration of hydronium ions is greater than 10⁻⁷ moles per litre).

In one embodiment the salt is a mixed salt that has the following formula, where HA and HB are different mono-protic acids:

| | |
|---|---|
| 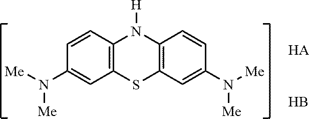 | when: p = 1 q = 1 n = 1 (1 + 1) × 1 = 2 |

However preferably the salt is not a mixed salt, and has the following formula:

| | |
|---|---|
| 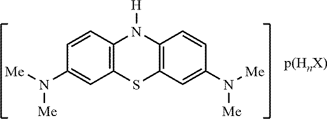 | when: p= 1, 2 n = 1, 2 p × n = 2 |

wherein each of HₙX is a protic acid, such as a di-protic acid or mono-protic acid.

In one embodiment the salt has the following formula, where H₂A is a di-protic acid:

| | |
|---|---|
| 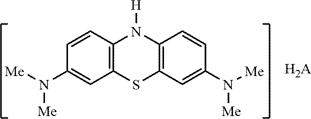 | when: p = 1 q = 0 n = 2 (1 + 0) × 2 = 2 |

Preferably the salt has the following formula which is a bis monoprotic acid:

| | |
|---|---|
| 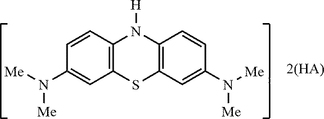 | when: p = 2 q = 0 n = 1 (2 + 0) × 1 = 2 |

Examples of protic acids which may be present in the LMTX compounds used herein include:
Inorganic acids: hydrohalide acids (e.g., HCI, HBr), nitric acid (HNO₃), sulphuric acid (H₂SO₄)
Organic acids: carbonic acid (H₂CO₃), acetic acid (CH₃COOH), methanesulfonic acid, 1,2-ethanedisulfonic acid, ethansulfonic acid, naphthalenedisulfonic acid, p-toluenesulfonic acid,
Preferred acids are monoprotic acid, and the salt is a bis(monoprotic acid) salt.

A preferred MT compound is LMTM:

| | | | |
|---|---|---|---|
| 1 | 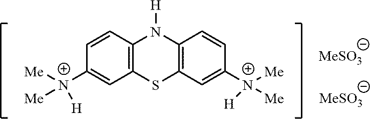 | LMT.2MsOH (LMTM) | 477.6 (1.67) |

### Weight Factors

The anhydrous salt has a molecular weight of around 477.6. Based on a molecular weight of 285.1 for the LMT core, the weight factor for using this MT compound in the invention is 1.67. By “weight factor” is meant the relative weight of the pure MT-containing compound vs. the weight of MT which it contains.

Other weight factors can be calculated for example MT compounds herein, and the corresponding dosage ranges can be calculated therefrom.

Other example LMTX compounds are as follows. Their molecular weight (anhydrous) and weight factor is also shown:

| | | | |
|---|---|---|---|
| 2 | 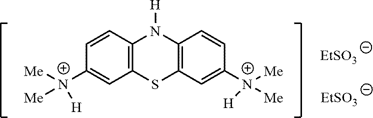 | LMT.2EsOH | 505.7 (1.77) |
| 3 | 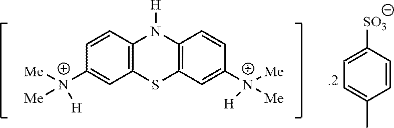 | LMT.2TsOH | 629.9 (2.20) |
| 4 | 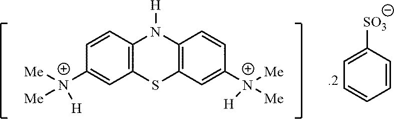 | LMT.2BSA | 601.8 (2.11) |
| 5 | 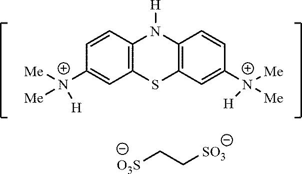 | LMT.EDSA | 475.6 (1.66) |

| | | | |
|---|---|---|---|
| 6 | 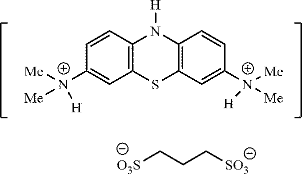 | LMT. PDSA | 489.6 (1.72) |
| 7 | 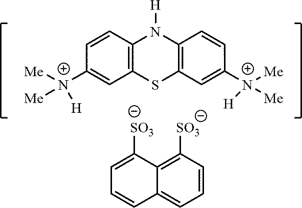 | LMT.NDSA | 573.7 (2.01) |
| 8 | 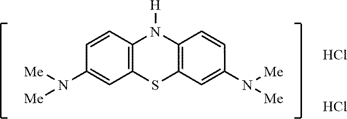 | LMT.2HCI | 358.33 (1.25) |

The dosages described herein with respect to MT thus apply mutatis mutandis for these MT-containing compounds, as adjusted for their molecular weight.

### Accumulation Factors

As will be appreciated by those skilled in the art, for a given daily dosage, more frequent dosing can lead to greater accumulation of a drug.

Therefore in certain embodiments of the claimed invention, the total daily dosed amount of MT compound may be relatively lower, when dosing more frequently (e.g. twice a day [bid] or three times a day [tid]), or higher when dosing once a day [qd].

### Treatment and Prophylaxis

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition.

The term “therapeutically-effective amount,” as used herein, pertains to that amount of a compound of the invention, or a material, composition or dosage from comprising said compound, which is effective for producing some desired therapeutic effect, commensurate with a reasonable benefit/risk ratio, when administered in accordance with a desired treatment regimen. The present inventors have demonstrated that a therapeutically-effective amount of an MT compound in respect of the diseases of the invention can be much lower than was hitherto understood in the art.

The invention also embraces treatment as a prophylactic measure.

The term “prophylactically effective amount,” as used herein, pertains to that amount of a compound of the invention, or a material, composition or dosage from comprising said compound, which is effective for producing some desired prophylactic effect, commensurate with a reasonable benefit/risk ratio, when administered in accordance with a desired treatment regimen.

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

### Combination Treatments and Monotherapy

The term “treatment” includes “combination” treatments and therapies, in which two or more treatments or therapies for COVID-19 are combined, for example, sequentially or simultaneously. These may be symptomatic or disease modifying treatments.

The particular combination would be at the discretion of the physician.

In combination treatments, the agents (i.e., an MT compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

An example of a combination treatment of the invention would be wherein the LMTX treatment is combined with chloroquine or hydroxychloroquine.

The dosage of chloroquine or hydroxychloroquine may be selected by the physician. Suggested protocols recommended for SARS-CoV-2 infection include a loading dose of 400 mg twice daily of hydroxychloroquine sulfate given orally, followed by a maintenance dose of 200 mg given twice daily for 4 days. An alternative is chloroquine phosphate when given 500 mg twice daily 5 days in advance (see e.g. Yao et al “In Vitro Antiviral Activity and Projection of Optimized Dosing Design of Hydroxychloroquine for the Treatment of Severe Acute Respiratory Syndrome Coronavirus 2 (SARS-CoV-2)” Clinical Infectious Diseases, 2020, Mar 9.

The MT-containing compound and the chloroquine or hydroxychloroquine may be administered sequentially within 12 hours of each other, or the subject may be pre-treated with one for a sustained period prior to treatment with the other, or the agents may be administered simultaneously, optionally within a single dosage unit.

As described herein, in relation to combination therapies, the invention provides methods of enhancing the therapeutic effectiveness of a first compound which is an MT compound at a dose described herein for the treatment of COVID-19, the method comprising administering to the subject a second compound, which second compound is chloroquine or hydroxychloroquine.

The invention further provides a first compound which is an MT compound at a dose described herein in a method of treatment of COVID-19 in a subject in a treatment regimen which additionally comprises treatment with a second compound, which second compound is chloroquine or hydroxychloroquine.

The invention further provides use of a compound which is chloroquine or hydroxychloroquine to enhance the therapeutic effectiveness of an MT compound at a dose described herein in the treatment of COVID-19 in the subject.

The invention further provides an MT compound at a dose described herein and chloroquine or hydroxychloroquine for use in a combination methods of the invention.

The invention further provides a compound which is chloroquine or hydroxychloroquine for use in a method of enhancing the therapeutic effectiveness of an MT compound at a dose described herein in the treatment of COVID-19 in a subject.

The invention further provides use of a first compound which is an MT compound at a dose described herein in combination with a second compound, which second compound is chloroquine or hydroxychloroquine, in the manufacture of a medicament for treatment of COVID-19.

The invention further provides use of an MT compound at a dose described herein in the manufacture of a medicament for use in the treatment of COVID-19, which treatment further comprises use of a second compound, which second compound is chloroquine or hydroxychloroquine.

The invention further provides use of chloroquine or hydroxychloroquine, in the manufacture of a medicament for use in the treatment of COVID-19 in a subject, which treatment further comprises use of an MT compound at a dose described herein and COVID-19.

Other combination treatments include the MT compounds with one or more of: lopinavir-ritonavir; arbidol; azithromycin, remdesivir, favipiravir, anti-inflammatory treatments such as actemra (tocilizumab), corticosteroids such as dexamethasone and other treatments such as convalescent plasma (see e.g. Thorlund, Kristian, et al. “A real-time dashboard of clinical trials for COVID-19.” The Lancet Digital Health (2020).

In other embodiments the treatment is a “monotherapy”, which is to say that the MT-containing compound is not used in combination (within the meaning discussed above) with another active agent for treating COVID-19 in the subject.

### Duration of Treatment

For treatment of COVID-19, a treatment regimen based on the low dose MT compounds will preferably extend over a sustained period of time appropriate to the disease and symptoms. The particular duration would be at the discretion of the physician.

For example, the duration of treatment may be:

1 to 14, e.g. 1, 2, 3, 4, 5, 6, 7, 8, 9, 10, 11, 12, 13 or 14 days.

1 to 4, e.g. 1, 2, 3 or 4 weeks.

For prophylaxis, the treatment may be ongoing.

In all cases the treatment duration will generally be subject to advice and review of the physician.

### Pharmaceutical Dosage Forms

The MT compound of the invention, or pharmaceutical composition comprising it, may be administered to the stomach of a subject/patient orally (or via a nasogastric tube) or intravenously.

Typically, in the practice of the invention the compound will be administered as a composition comprising the compound, and a pharmaceutically acceptable carrier or diluent.

In some embodiments, the composition is a pharmaceutical composition (e.g., formulation, preparation, medicament) comprising a compound as described herein, and a pharmaceutically acceptable carrier, diluent, or excipient.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some embodiments, the composition is a pharmaceutical composition comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In some embodiments, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington’s Pharmaceutical Sciences, 20th edition, pub. Lippincott, Williams & Wilkins, 2000; and Handbook of Pharmaceutical Excipients, 2nd edition, 1994.

One aspect of the present invention utilises a dosage unit (e.g., a pharmaceutical tablet or capsule) comprising an MT compound as described herein (e.g., obtained by, or obtainable by, a method as described herein; having a purity as described herein; etc.), and a pharmaceutically acceptable carrier, diluent, or excipient.

The “MT compound”, although present in relatively low amount, is the active agent of the dosage unit, which is to say is intended to have the therapeutic or prophylactic effect in respect of COVID-19. Rather, the other ingredients in the dosage unit will be therapeutically inactive e.g. carriers, diluents, or excipients.

Thus, preferably, there will be no other active ingredient in the dosage unit, no other agent intended to have a therapeutic or prophylactic effect in respect of a disorder for which the dosage unit is intended to be used, other than in relation to the combination treatments described herein.

In some embodiments, the dosage unit is a tablet.

In some embodiments, the dosage unit is a capsule.

In some embodiments, said capsules are gelatine capsules.

In some embodiments, said capsules are HPMC (hydroxypropylmethylcellulose) capsules.

The appropriate quantity of MT in the composition will depend on how often it is taken by the subject per day.

An example dosage unit may contain 10 to 30 mg of MT.

In some embodiments, the amount is about 10, 11, 12, 13, 14, 15, 16, 17, 18, 19, 20, 21, 22, 23, 24, 25, 26, 27, 28, 29, or 30 mg of MT.

Using the weight factors described or explained herein, one skilled in the art can select appropriate amounts of an MT-containing compound to use in oral formulations.

As explained above, the MT weight factor for LMTM is 1.67. Since it is convenient to use unitary or simple fractional amounts of active ingredients, non-limiting example LMTM dosage units may include 17 mg etc.

In one embodiment there is provided a dosage unit pharmaceutical composition which comprises about 17, 27, 34 mg etc. of LMTM.

### Subjects, Patients and Patient Groups

In some embodiments the subject may be a human who has been diagnosed as having (“confirmed”) COVID-19, or wherein said method comprises making said diagnosis.

Diagnosis of COVID-19 may be via any method known in the art. Examples include laboratory testing for the presence of the SARS-CoV-2 virus - for example directly based on the presence of virus itself (e.g. using RT-PCR and isothermal nucleic acid amplification, or the presence of antigenic proteins) or indirectly via antibodies produced in response to infection. Other methods of diagnosis include chest X-ray, optionally in combination with characteristic symptoms as described below (see e.g. Li, Xiaowei, et al. “Molecular immune pathogenesis and diagnosis of COVID-19.” Journal of Pharmaceutical Analysis (2020); Fang, Yicheng, et al. “Sensitivity of chest CT for COVID-19: comparison to RT-PCR.” Radiology (2020): 200432; Chan, Jasper Fuk-Woo, et al. “Improved Molecular Diagnosis of COVID-19 by the Novel, Highly Sensitive and Specific COVID-19-RdRp/Hel Real-Time Reverse Transcription-PCR Assay Validated In Vitro and with Clinical Specimens.” Journal of Clinical Microbiology 58.5 (2020); Tang, Yi-Wei, et al. “The laboratory diagnosis of COVID-19 infection: current issues and challenges.” Journal of Clinical Microbiology (2020).

In some embodiment the subject is a human who has been assessed as being “at risk” of, COVID-19, or having probable COVID-19 e.g. based on situational or other data.

Those are particular risk of COVID-19 include:
People who have been in close contact with one or more COVID-19 cases
People 65 years and older;
People who live in a nursing home, care home, or long-term care facility;
People of all ages with relevant underlying medical conditions, particularly if not well controlled, including:
   People with chronic lung disease or moderate to severe asthma People who have serious heart conditions People who are immunocompromised
      As is known in the art, many conditions can cause a person to be immunocompromised, including cancer treatment, smoking, bone marrow or organ transplantation, immune deficiencies, poorly controlled HIV or AIDS, and prolonged use of corticosteroids and other immune weakening medications
   People with severe obesity (body mass index [BMI] of 40 or higher)
   People with diabetes
   People with chronic kidney disease undergoing dialysis
   People with liver disease

Symptoms or circumstances which are indicative of potential (“probable”) COVID-19 include:
1) a patient with acute respiratory tract infection (sudden onset of at least one of the following: cough, fever, shortness of breath) AND with no other aetiology that fully explains the clinical presentation AND with a history of travel or residence in a country/area reporting local or community transmission during the 14 days prior to symptom onset; OR
2) a patient with any acute respiratory illness AND having been in close contact with a confirmed or probable COVID-19 case in the last 14 days prior to onset of symptoms; OR
3) A patient with severe acute respiratory infection (SARI) (fever and at least one sign/symptom of respiratory disease (e.g., cough, fever, shortness breath)) AND requiring hospitalisation AND with no other aetiology that fully explains the clinical presentation.

“Close contact” as used herein is defined as:
A person living in the same household as a COVID-19 case;
A person having had direct physical contact with a COVID-19 case (e.g. shaking hands);
A person having unprotected direct contact with infectious secretions of a COVID-19 case (e.g. being coughed on, touching used paper tissues with a bare hand);
A person having had face-to-face contact with a COVID-19 case within 2 metres and > 15 minutes;
A person who was in a closed environment (e.g. classroom, meeting room, hospital waiting room, etc.) with a COVID-19 case for 15 minutes or more and at a distance of less than 2 metres;
A healthcare worker (HCW) or other person providing direct care for a COVID-19 case, or laboratory workers handling specimens from a COVID-19 case without recommended personal protective equipment (PPE) or with a possible breach of PPE;
A contact in an aircraft sitting within two seats (in any direction) of the COVID-19 case, travel companions or persons providing care, and crew members serving in the section of the aircraft where the index case was seated (if severity of symptoms or movement of the case indicate more extensive exposure, passengers seated in the entire section or all passengers on the aircraft may be considered close contacts).

The epidemiological link to a probable or confirmed case may have occurred within a 14-day period before the onset of illness in the suspected case under consideration.

Given the overlap in the population characteristics between those at risk of AD and COVID-19 (for example care home populations), and the safety of LMTX in this at-risk population, the treatments of the present invention may in principle be performed in conjunction with treatments for the purpose of AD.

The patient may be an adult human, and the population-based dosages described herein are premised on that basis (typical weight 50 to 70 kg). If desired, corresponding dosages may be utilised for subjects falling outside of this range by using a subject weight factor whereby the subject weight is divided by 60 kg to provide the multiplicative factor for that individual subject.

### Labels, Instructions and Kits of Parts

The unit dosage compositions described herein (e.g. a low dose MT-containing compound plus optionally other ingredients, or MT composition more generally for treatment in AD) may be provided in a labelled packet along with instructions for their use.

In one embodiment, the pack is a bottle, such as are well known in the pharmaceutical art. A typical bottle may be made from pharmacopoeial grade HDPE (High-Density Polyethylene) with a childproof, HDPE pushlock closure and contain silica gel desiccant, which is present in sachets or canisters. The bottle itself may comprise a label, and be packaged in a cardboard container with instructions for us and optionally a further copy of the label.

In one embodiment, the pack or packet is a blister pack (preferably one having aluminium cavity and aluminium foil) which is thus substantially moisture-impervious. In this case the pack may be packaged in a cardboard container with instructions for us and label on the container.

Said label or instructions may provide information regarding COVID-19 or SARS-CoV-2.

### Methods of Treatment

Another aspect of the present invention, as explained above, pertains to a method of treatment of COVID-19 comprising administering to a patient in need of treatment a prophylactically or therapeutically effective amount of a compound as described herein, preferably in the form of a pharmaceutical composition.

### Use in Methods of Therapy

Another aspect of the present invention pertains to a compound or composition as described herein, for use in a method of treatment of COVID-19 of the human or animal body by therapy.

### Use in the Manufacture of Medicaments

Another aspect of the present invention pertains to use of an MT compound or composition as described herein, in the manufacture of a medicament for use in treatment of COVID-19.

In some embodiments, the medicament is a composition e.g. a low-dose unit dose composition as described herein.

### Mixtures of Oxidised and Reduced MT Compounds

The LMT-containing compounds utilised in the present invention may include oxidised (MT⁺) compounds as ‘impurities’ during synthesis, and may also oxidize (e.g., autoxidize) after synthesis to give the corresponding oxidized forms. Thus, it is likely, if not inevitable, that compositions comprising the compounds of the present invention will contain, as an impurity, at least some of the corresponding oxidized compound. For example an “LMT” salt may include up to 15% e.g. 10 to 15% of MT⁺ salt.

When using mixed MT compounds, the MT dose can be readily calculated using the molecular weight factors of the compounds present.

### Salts and Solvates

Although the MT-containing compounds described herein are themselves salts, they may also be provided in the form of a mixed salt (i.e., the compound of the invention in combination with another salt). Such mixed salts are intended to be encompassed by the term “and pharmaceutically acceptable salts thereof”. Unless otherwise specified, a reference to a particular compound also includes salts thereof.

The compounds of the invention may also be provided in the form of a solvate or hydrate. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, a penta-hydrate etc. Unless otherwise specified, any reference to a compound also includes solvate and any hydrate forms thereof.

Naturally, solvates or hydrates of salts of the compounds are also encompassed by the present invention.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1: computational chemistry modelling of the high affinity LMT/MT⁺-heme interaction.

### REFERENCE EXAMPLE 1 - METHYLTHIONINIUM CHLORIDE (MTC) AS AN ANTIVIRAL

MTC (methylthioninium chloride, methylene blue) has been available as a drug since 1876. It is on the world health organisation’s list of essential medicines, which is a list of the safest and most effective medicines in a health system.

Several studies have investigated the antiviral activity of MTC. One such study concluded that 23 out of 36 enrolled hepatitis C patients had a decrease in viral counts of between 70-100% following a dosage regiment of 130 mg/MTC per day (ie 98 mg/MT-equivalent per day) for 50 days. 12 patients (52%) had between 0.7-1 log reduction in viral load, 6 (26%) had between 1-2 log reduction in viral load and 5 (22%) had viral clearance. These uncorroborated results suggest that MTC may have useful activity in treating hepatitis C (Wood et al., 2006; Mehta et al., 2006).

One potential mechanism which has been proposed by which MTC may exert, or enhance, an anti-viral effect in vivo is via nucleic acid intercalation (see Jamison, J. M., et al. “RNA-Intercalating Agent Interactions: in vitro Antiviral Activity Studies.” Antiviral Chemistry and Chemotherapy 1.6 (1990): 333-347).

Further support comes from the routine use of photoactivated MTC for viral sterilisation of blood products. Viruses susceptible to MTC treatment include HIV-1 and 2, herpes, hepatitis C, and toga viruses (Muller-Breitkreutz 1998, Mohr, 1999).

In a very recent report, the rate of infection with SARS-CoV-2 was examined retrospectively in a cohort of 2,500 vulnerable patients receiving MTC at an oral dose of 315 mg/MTC per day (236 mg/MT-equivalent per day) as part of their routine cancer chemotherapy regime. This cohort came from a database of 30,000 people undergoing metabolic treatment with lipoic acid/hydroxycitrate. As of 27 Mar. 2020, none of those receiving MTC had clinical symptoms consistent with COVID-19 (Henry et al., 2020). The paper does not, however, report the frequency of cases in patients not receiving MTC. Nevertheless, on the basis of this result, the authors are reported to have initiated an open prospective single centre study of MTC at a dose of 150 mg/MTC per day (113 mg/MT-equivalent per day) in patients with a clinical diagnosis of COVID-19 (https://guerir-du-cancer.fr/essai-ouvert-testant-le-bleu-de-methylene-dans-le-covid-19/).

### Reference Example 2 - Chloroquine/Hydroxychloroquine as an Antiviral

Independently of MTC, the antimalarial compound chloroquine and the related hydroxychloroquine are currently being investigated globally to assess their effectiveness as antiviral drugs against SARS-CoV-2.

Several studies have shown the effectiveness of chloroquine against SARS-CoV in vitro (Vincent 2005, Keyaerts 2004). More recently, this has also been shown for SARS-CoV-2 (Liu et al., 2020). Researchers in France have published a study in which they treated 20 COVID-19 patients with hydroxychloroquine. They concluded that the drug significantly reduced viral load in nasal swabs (Gautret et al., 2020).

In a review of the currently available evidence (Cortegiani et al., 2020), the authors concluded that clinical research use of chloroquine was justified in patients with COVID-19, although this should be restricted to ethics-approved trials or under the Monitored Emergency Use of Unregistered Interventions framework. However, according to a news report, a recent study appears to have found no benefit in advanced cases, with similar mortality rates in patients treated or not treated with chloroquine (https://www.scmp.com/news/china/science/article/3080055/anti-malarial-treatment-hailed-trump-has-no-benefit-coronavirus).

Others have reported that a beneficial effect may arise through anti-inflammatory properties and recovery of lymphopenia (Tang, Wei, et al. “Hydroxychloroquine in patients with COVID-19: an open-label, randomized, controlled trial.” medRxiv (2020).

Researchers have also reported positive effects of treatment on reduction of exacerbation of pneumonia, improvement in lung imaging findings, promotion of conversion to a virus-negative status and shortening of disease course, although data are unavailable (Gao et al., 2020). Experts from the National Health Commission for the People’s Republic of China reviewed the available data and recommended inclusion of choloroquine in forthcoming guidelines.

The mechanism by which the antimalarial compounds chloroquine/hydroxychloroquine should have potential activity against SARS-CoV-2 virus is unknown.

### Example 3 - Hydromethylthionine Salts as a Monotherapy for COVID 19

The methylthioninium (MT) moiety can exist in the oxidised MT⁺ form and in the reduced LMT form:

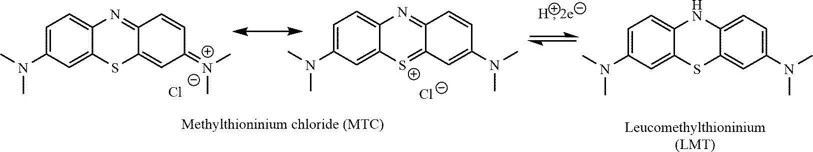

MTC is the chloride salt of the oxidised MT⁺ form. It needs to be converted to the LMT form by a thiazine dye reductase activity in the gut to permit absorption and distribution to deep compartments including red cells and brain (Baddeley et al., 2015). Likewise, in isolated red cell preparations, MT⁺ needs to be converted to LMT to permit cell uptake (May et al., 2004).

WO2007/110627 disclosed certain 3,7-diamino-10H-phenothiazinium salts, effective as drugs or pro-drugs for the treatment of diseases including Alzheimer’s disease and other diseases such as Frontotemporal dementia (FTD), as well as viral diseases generally. These compounds are also in the “reduced” or “leuco” form when considered in respect of MTC. These leucomethylthioninium compounds were referred to herein as “LMTX” salts.

WO2012/107706 described other LMTX salts having superior properties to the LMTX salts listed above, including leuco-methylthioninium bis(hydromethanesulfonate) (LMTM) (WHO INN designation: hydromethylthionine):

| | |
|---|---|
| 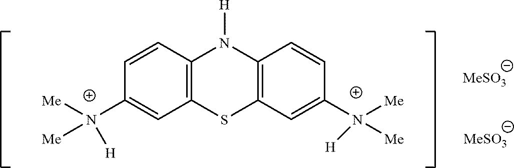 | N,N,N′,N′-tetramethyl-10H-phenothiazine-3,7-diaminium bis(hydromethanesulfonate). |
| | LMT.2MsOH / LMTM |

Synthesis of LMTX and LMTM compounds can be performed according to the methods described in these publications, or a method analogous to those.

LMTM is in development for treatment of Alzheimer’s disease (AD) and related neurodegenerative disorders (Gauthier et al., 2016; Wilcock et al., 2018; Schelter et al., 2019; Shiells et al., 2020). A global clinical trial in AD is currently ongoing using the dose (16 mg/day) shown to have optimal activity on clinical and neuroimaging endpoints in AD (Schelter et al., 2019).

MTC was previously the focus of a potential treatment for AD because of its ability to block pathological aggregation of the microtubule associated protein tau which forms neurofibrillary tangles and is responsible for clinical dementia in AD (Wischik et al., 1996; Harrington et al., 2015). A Phase 2 dose-finding study with MTC identified 138 mg/day as the minimum effective dose (Wischik et al., 2015).

Because LMT absorption from LMTM is much more efficient, the minimum effective dose required for anti-dementia effects was found to be 8 mg/day, and 16 mg/day was found to be the optimally effective dose (Schelter et al., 2019). This is due to a more than 60-fold better brain:plasma ratio for LMTM resulting from rapid uptake into red cells and distribution to deep compartment tissues. Free plasma LMT is subject to efficient first-pass metabolism which converts it to an inactive conjugate and which is the predominant species in plasma. LMTM also has 20-fold better uptake into red cells when administered intravenously (Baddeley et al., 2015).

It should be noted that once absorbed into cells LMT will be present is equilibrium with MT+, with the balance depending on the availability of reducing equivalents in the cell.

The potential for LMT compounds to be active at the low dose, and the apparent lack of a dose-response, are discussed in WO2018/019823 and it is hypothesised that there may be a critical threshold for activity at the tau aggregation inhibitor target, and that the effect of higher doses may plateau or may even become negative at brain concentrations above 1 µM. WO2020/020751 indicates that a plasma concentration of 0.5-1.0 ng/mL is desirable for treatment of AD.

The oral doses of MTC which appear to have anti-viral activity are in the range 100 - 236 mg/MT per day. Using the activity data in AD as a basis for comparison, we have calculated that this would be equivalent to doses of LMTM in the range 12 - 27 mg/MT per day. This is in the same dosage range (16 mg/MT-equivalent per day) as required for optimal activity in AD. This suggests that similar concentrations of LMT at the site of action are required for clinical anti-viral and anti-dementia pharmacological activity. We expect the high dose of MTC (236 mg/MT-equivalent per day) reported by Henry et al. is unlikely to have been absorbed adequately (Baddeley et al., 2015), consistent with a lower LMTM dose requirement. Therefore, the oral dose of 16 mg/MT-equivalent per day would be a suitable treatment for anti- SARS-CoV-2 activity in COVID-19 patients. Intravenous LMTM doses required to achieve the desired trough concentrations of 0.5-1.0 ng/mL have been predicted using PK parameters from 1475 patients with either Alzheimer’s Disease or behavioral variant frontotemporal dementia who had received LMTM orally in previous Phase 3 trials (see also WO2020/020751).

These PK parameters after oral dosing were scaled to IV dosing by multiplying the individual values by 0.75 to account for the assumed 75% systemic bioavailability of the oral formulation. A simple two-compartment model was then employed to predict drug concentrations over time for various dosing regimens.

When given via continuous infusion, an infusion rate of 0.6 mg/hr is predicted to result in 95.5% of subjects achieving steady-state concentrations of above 0.5 ng/mL with 8.8% having steady-state concentrations above 1.0 ng/mL.

Thus when given as intermittent bolus doses administered over 5 minutes, a dose of 4.4 mg every six hours is predicted to result in steady-state trough concentrations above 0.5 ng/mL in 95.4% of subjects with 6.2% having steady-state concentrations above 1.0 ng/mL.

Three Phase 3, double-blind, controlled studies of LMTM have been completed (one each in subjects with mild and mild to moderate AD and one in subjects with bvFTD). Results of the AD studies have been published (Gauthier et al., 2016; Wilcock et al., 2018; Shiells et al., 2020). These studies provide an overview of the more common adverse events that might be expected at a dose of LMTM 16 mg/day. In these three studies, 1897 subjects received at least one dose of LMTM. Of these, 860 subjects received LMTM 8 mg/day and 1037 subjects received at least one dose of LMTM in the higher doses of 150 to 250 mg/day. The mean ages of study participants were 71 years (ranging up to 89 years) for subjects with AD and 63 years (ranging up to 79 years) for subjects with bvFTD.

The overall person-years of exposure to LMTM 8 mg/day was 995.2 person-years and to the higher LMTM doses of 150 to 250 mg/day was 988.6 person-years. Six percent (6%) of the subjects discontinued LMTM 8 mg/day due to adverse events; the proportion of subjects discontinuing due to adverse events in the higher dose groups was higher (14%).

The most common Treatment-Emergent Adverse Events (TEAEs) considered at least possibly associated with LMTM given in a dose of 8 mg/day were gastrointestinal (mostly diarrhoea and nausea), genitourinary (mostly pollakiuria and urinary incontinence), haematologic (anaemia, decreased folate, and folate deficiency), and nervous system related (mostly fatigue, dizziness, headache, agitation, and insomnia). At the higher LMTM doses studied, 150 to 250 mg/day, there was a dose-related increase in the incidence of anaemia-related TEAEs (decreased haemoglobin in addition to anaemia, decreased folate, and folate deficiency), gastrointestinal events (including vomiting and diarrhoea), and genitourinary events (including dysuria, micturition urgency, and apparent urinary tract infections in addition to pollakiuria and urinary incontinence). The lack of a dose response in falls and nervous system/psychiatric events (other than agitation) suggests that these are associated with the subjects’ underlying condition rather than treatment.

Haematological parameters showed dose-dependent decreases in RBC count, haemoglobin, and haematocrit with greater decreases in the higher dose groups compared to LMTM 8 mg/day, which showed minimal change. No clinically meaningful trends were observed based on vital sign measurements, ECGs, or the C-SSRS.

In summary, therefore, the safety of LMTM has been studied in over 2400 patients in Phase 1 and Phase 3 trials at repeat doses up to 450 mg/day. Even at the highest doses examined, the safety profile of LMTM remains benign and consistent with further clinical development.

There is evidence that chloroquine and LMT both act in a similar manner as anti-malarial agents (Atamna et al., 1996; Blank et al., 2012). Oxidation of haemoglobin to form metHb, which is required for parasite maturation is dependent on rendering the iron-porphyrin ring non-toxic. The parasite does this by forming haemazoin polymers from haematin (porphyrin-Fe3⁺). Both chloroquine and LMT form complexes with haematin which prevent its polymerisation, thereby leaving haematin to remain toxic for the parasite following digestion of haemoglobin within its food vacuoles.

There has been recent interest in the proposal that heme-binding of SARS-CoV-2 proteins may impair blood oxygen-carrying capacity. This is discussed in a (non-peer-reviewed) computational modelling report (Liu and Li, 2020) which has since been shown to be technically flawed in a (non-peer-reviewed) critique (Read, 2020).

Nevertheless, there is some evidence supporting a role for red cells in COVID-19. Macaques showed decrease in red blood cell numbers following infection with SARS-CoV-2 (Munster et al., 2020). It is has been reported that susceptibility to SARS-CoV-2 appears to be determined by blood group (Yang et al., 2020). In the Chen et al. (2020) report on COVID-19 patients in Wuhan, there was an elevation in serum ferritin and increase in total bilirubin. Elevation in ferritin levels can occur as a result of dissociation of iron from heme (Sassa, 2006) and increased bilirubin is associated with ineffective erythropoiesis (Trier et al., 2013). However, elevation of ferritin levels could also be the result of macrophage activation syndrome, and there appears to be less haemolysis in COVID-19 patients than seen in influenza infections (Emmenegger et al. 2002; Huang et al., 1981). Abrahams (2020) argues in another (non-peer-reviewed) opinion piece that some of the haematological features of SARS-CoV-2 resemble acute porphyria. This could explain the neurovisceral and neurological symptoms seen in both porphyria (Pischik and Kauppinen, 2015; Sassa, 2006) and in up to 50% of COVID-19 patients (Poggiali et al., 2020; Zhao et al., 2020; Mao et al. 2020).

MTC has been used since the 1930’s for treatment of methaemoglobinemia and cyanide poisoning, and remains the standard treatment for these conditions. In methaemoglobinemia, the heme iron is in the ferric (Fe³⁺) state as opposed to the normal ferrous state (Fe²⁺) and therefore cannot bind oxygen efficiently (Curry et al., 1982). MTC is typically given intravenously at a dose of 1-2 mg/kg, and is associated with rapid clinical improvement and resolution of methemoglobinemia.

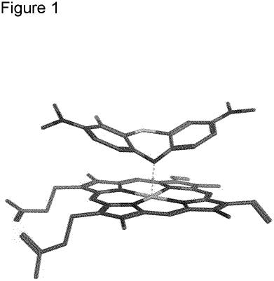

The mode of action of LMT in malaria and methaemoglobinaemia are very similar. In both, the oxidised MT⁺ form of methylthionine given as MTC is first reduced to LMT at the cell surface as a prerequisite for red cell entry (May et al., 2004). It is then LMT which is the active species at the heme site, binding to porphyrin and permitting the transfer of an electron which converts Fe³⁺ to Fe²⁺, generating MT⁺ in the process (Yubisui et al., 1980; Blank et al., 2012). MT⁺ is then converted back to LMT by nicotinamide adenine dinucleotide phosphate and other reducing equivalents which are subject to continuous regeneration via glycolysis within the red cell. Computational chemistry modelling shown in FIG. 1 provides a structural basis explaining the dynamics of the high affinity LMT/MT⁺-heme interaction. The LMT nitrogen orientates itself towards the Fe³⁺ of the heame porphyrin within 2.1Å (dotted line in FIG. 1). This close interaction then facilitates the transfer of an electron from LMT to the Fe³⁺, thereby reducing it to Fe²⁺ and the resulting formation of MT⁺.

The present inventors have noted that the ability to interact with the porphyrin core of haemoglobin is common to both chloroquine and LMT. Chloroquine is known to induce the release of tissue-bound porphyrins and it has been shown that following chloroquine administration to porphyria cutanea tarda (PCT) patients, the initial event is release and rapid elimination of bound hepatic porphyrin (Scholnick et al., 1973).

Regardless of this, the inventors propose that complexation with heme by LMT delivered as LMTX can provide another intervention, over and above direct anti-viral action, into the aetiology of COVID19.

LMT has a redox potential close to zero which is mid-way between the potentials of Complex I and Complex IV in the mitochondrial electron transport chain. It thus has the ability to enhance mitochondrial function by acting as an electron shuttle (Atamna & Kumar 2010). Consistent with this, LMTM has been confirmed recently to enhance brain Complex IV activity in a tau transgenic mouse model (Riedel et al., 2020). This activity translates into an anti-ischaemic activity which limits the extent of infarction in a unilaterally ligated rat-brain model of cerebral ischaemia (Rodriguez et al., 2014).

The inventors propose that, since LMT is distributed rapidly into deep compartments following dosing with LMTX, it may be used to enhance mitochondrial function in many tissues in the event of limited oxygen delivery. Thus this can provide a further intervention into the aetiology of COVID19.

In addition to enhancing mitochondrial function, the MTC dosed orally has been shown to increase mitochondrial biogenesis (Stack et al., 2014). Enhancement of mitochondrial biogenesis is linked to the ability to increase in Nrf2 levels (Gureev et al., 2016). Rojo de la Vega and colleagues argue in an extensive review of the research literature that Nrf2 plays an important protective role with respect to oxidative and inflammatory lung damage in Acute Lung Injury / Acute Respiratory Distress Syndrome (ADI/ARDS) (Rojo de al Vega et al., 2016).They present evidence to show that pharmacological activation of Nrf2 would be expected to ameliorate alveolar damage not only resulting from primary infection but also from mechanical and hyperoxic injury resulting from Ventilation Induced Lung Injury (VILI). Oral dosing with MTC at 30 mg/kg has been shown to increase Nrf2 levels in brain (Stack et al., 2014). As in red cells, the oxidised MT⁺ needs to be reduced to LMT to permit uptake into pulmonary endothelial cells (Merker et al., 1997).

The inventors propose that, since LMT has the potential to induce Nrf2 in ADI/ARDS, LMTX may be used to ameliorate alveolar damage. Thus this can provide a further intervention into the aetiology of COVID19.

### Example 4 - Hydromethylthionine Salts as Treatment for COVID-19

For the foregoing rationale the LMTX class of compounds may provide benefits in the treatment (including prophylactic treatment) of COVID-19 patients both alone and in combination with chloroquine (or analogues thereof e.g. hydroxychloroquine).

To summarise, LMTX can provide benefits to subjects in (1) permitting reduction of viral load, (2) complexation with heme which may, either directly or indirectly, provide supportive activity in COVID-19, (3) mitigate damage to pulmonary endothelium resulting from inflammatory, hyperoxic and mechanical injury to lung.

Furthermore, the LMTM does not have the cardiotoxicity that limits the dose and duration of treatment with chloroquine/hydroxychloroquine, and may therefore provide a safer approach to treatment either alone or in combination with that agent.

An appropriate dosage of MT which is appropriate to all of these aims is around 10-30 mg/MT p.o. per day, for example 15 or 16 mg/MT-equivalent per day, as required for optimal activity in AD.

For IV dosing, around 10 to 25 mg of MT, more preferably 4 and 20 mg of MT to the subject per day, is preferred.

### REFERENCES

Abrahams L. Covid-19: acquired acute porphyria hypothesis. 2020. 10.31219/osf.io/4wkfy

Atamna H, Krugliak M, Shalmiev G, Deharo E, Pescarmona G, Ginsburg H. Mode of antimalarial effect of methylene blue and some of its analogues on Plasmodium falciparum in culture and their inhibition of P. vinckei petteri and P. yoelii nigeriensis in vivo. Biochem Pharmacol. 1996. 51(5):693-700

Baddeley TC, McCaffrey J, Storey JMD, Cheung JKS, Melis V, Horsley D, Harrington CR, Wischik CM. Complex disposition of methylthioninium redox forms determines efficacy in tau aggregation inhibitor therapy for Alzheimer’s disease. J Pharmacol Exp Ther. 2015. 352, 110-118.

Blank O, Davioud-Charvet E, Elhabiri M. Interactions of the antimalarial drug methylene blue with methemoglobin and heme targets in Plasmodium falciparum: a physico-biochemical study. Antioxid Redox Signal. 2012. 17(4):544-54

Brent, J., Burkhart, K., Dargan, P., Hatten, B., Megarbane, B., Palmer, R., & White, J. (Eds.). Critical care toxicology: diagnosis and management of the critically poisoned patient. 2017.

Chen N, Zhou M, Dong X, Qu J, Gong F et al., Epidemiological and clinical characteristics of 99 cases of 2019 novel coronavirus pneumonia in Wuhan, China: a descriptive study. The Lancet. 2020. 395 (10223): 507-513

Cortegiani A, Ingoglia G, Ippolito M, Giarratano A, Einav S. A systematic review on the efficacy and safety of chloroquine for the treatment of COVID-19. J Crit Care. 2020

Curry S. Methemoglobinemia. Ann Emerg Med. 1982. 2:214-21

Davis TM, Syed DA, Barrett PH. Toxicity related to chloroquine treatment of resistant vivax malaria. Ann Pharmacother. 2003. 37(4):526-9.

Fall B, Camara C, Fall M, Nakoulima A, Dionne P, Diatta B, Dieme Y, Wade B, Pradines B. Plasmodium falciparum susceptibility to standard and potential anti-malarial drugs in Dakar, Senegal, during the 2013-2014 malaria season. Malar J. 2015. 14, 60.

Gauthier S, et al. Efficacy and safety of tau-aggregation inhibitor therapy in patients with mild or moderate Alzheimer’s disease: a randomised, controlled, double-blind, parallel-arm, phase 3 trial. Lancet. 2016. 388, 2873-2884.

Gautret P, Lagier JC, Parola P, et al. Hydroxychloroquine and azithromycin as a treatment of COVID-19: results of an open-label non-randomized clinical trial [published online ahead of print, 2020 Mar 20]. Int J Antimicrob Agents. 2020. 105949

Gao J, Zhenxue T, Yang X. Breakthrough: Chloroquine phosphate has shown apparent efficacy in treatment of COVID-19 associated pneumonia in clinical studies. BioScience Trends. 2020. 14(1):72-73

Harrington CR, Storey JM, Clunas S, et al. Cellular Models of Aggregation-dependent Template-directed Proteolysis to Characterize Tau Aggregation Inhibitors for Treatment of Alzheimer Disease. J Biol Chem. 2015. 290(17): 10862-10875

Henry M, Summa M, Patrick L, Schwartz L. A cohort of cancer patients with no reported cases of SARS-CoV-2 infection: the possible preventive role of Methylene Blue. Substantia. 2020. 4(1)

Keyaerts E, Vijgen L, Maes P, Neyts J, Van Ranst M. In vitro inhibition of severe acute respiratory syndrome coronavirus by chloroquine. Biochem Biophys Res Commun. 2004. Oct 8;323(1):264-8.

Krogstad DJ, Schlesinger PH. The basis of antimalarial action: non-weak base effects of chloroquine on acid vesicle PH. Am J Trop Med Hyg. 1987. 36(2):213-20

Liu, J., Cao, R., Xu, M. et al. Hydroxychloroquine, a less toxic derivative of chloroquine, is effective in inhibiting SARS-CoV-2 infection in vitro. Cell Discov. 2020. 6, 16

Liu W & Li H. COVID-19: Attacks the 1-Beta Chain of Hemoglobin and Captures the Porphyrin to Inhibit Human Heme Metabolism. 2020. ChemRxi

Mao L, Wang M, Chen S, He Q, Chang J, Hong C, Zhou Y, Wang D, Li Y, Jin H & Hu B. Neurological manifestations of hospitalised patients with COVID-19 in Wuhan, China: a retrospective case series study. 2020. medRxiv

May JM, Qu ZC, Cobb CE. Reduction and uptake of methylene blue by human erythrocytes. American Journal of physiology. 2004. 286(6):C1390-8

Mehta G, Mawdsley A et al., the effect of oral methylene blue on viral load in chronic hepatitis C infection. Poster presented at British association for the study of the liver (BASL) meeting. 2006 Sept. Dublin, Ireland.

Mohr, H. Virus inactivation of plasma by methylene blue/ light exposure. Pediatr Res. 1999. 45, 946

Muller-Breitkreutz K, Mohr H. Hepatitis C and human immunodeficiency virus RNA degradation by methylene blue/light treatment of human plasma. J Med Virol. 1998. 56(3):239-45.

Munster VJ, Feldmann F, et al. Respiratory disease and virus shedding in rhesus macaques inoculated with SARS-CoV-2. 2020. bioRxiv.

Pischik E & Kauppinen R. An update of clinical management of acute intermittent porphyria. The Application of Clinical Genetics. 2015. 8: 201

Poggiali E, Ramos P, Bastoni D, Vercelli A, Magnacavallo A. Abdominal Pain: A Real Challenge in Novel COVID-19 Infection. European Journal of Case Reports in Internal Medicine. 2020. 7(4)

Rengelshausen J, Burhenne J, Frohlich M, Tayrouz Y, Singh SK, Riedel KD, Müller O, Hoppe-Tichy T, Haefeli WE, Mikus G, Walter-Sack I. Pharmacokinetic interaction of chloroquine and methylene blue combination against malaria. Eur J Clin Pharmacol. 2004. 60(10):709-15

Riedel, G, Klein, J, Niewiadomska, G, Kondak, C, Schwab, K et al. (2019) Mechanisms of anticholinesterase interference with tau aggregation inhibitor activity in a tau-transgenic mouse model. Curr. Alzheimer Res. 2020. 17(3): http://dx.doi.org/10.2174/1567205017666200224120926

Schelter BO, Shiells HC, Baddeley TC, et al., Concentration-dependent activity of hydromethylthionine on cognitive decline and brain atrophy in mild to moderate Alzheimer’s disease. J Alzheimers Dis. 2019. 72, 931-946

Shiells, Helen et al. ‘Concentration-Dependent Activity of Hydromethylthionine on Clinical Decline and Brain Atrophy in a Randomized Controlled Trial in Behavioral Variant Frontotemporal Dementia’. JAlzheimers Dis. 2020. DOI 10.3233/JAD-191173

Sassa S. Modern diagnosis and management of the porphyrias. British Journal of Haematology. 2006. 135: 281-29

Scholnick PL, Epstein J & Marver HS. The Molecular Basis of the Action of Chloroquine in Porphyria Cutanea Tarda. Journal of Investigative Dermatology. 1973. 61 (4): 226-232

Trier H, Krishnasamy VP & Kasi PM. Clinical manifestations and diagnostic challenges in acute porphyrias. 2013 Case Reports in Hematology

Vincent MJ, Bergeron E, Benjannet S, et al. Chloroquine is a potent inhibitor of SARS coronavirus infection and spread. Virol J. 2005. 2:69

Wilcock GK, Gauthier S, Frisoni GB, et al. Potential of Low Dose Leuco-Methylthioninium Bis(Hydromethanesulphonate) (LMTM) Monotherapy for Treatment of Mild Alzheimer’s Disease: Cohort Analysis as Modified Primary Outcome in a Phase III Clinical Trial. J Alzheimers Dis. 2018. 61(1):435-457

Wischik CM, Edwards PC, Lai RYK, Roth M, Harrington CR (1996) Selective inhibition of Alzheimer disease-like tau aggregation by phenothiazines. Proc Natl Acad Sci USA 93, 11213-11218.

Wischik CM, Staff RT, Wischik DJ, Bentham P, Murray AD, Storey JMD, Kook KA, Harrington CR (2015) Tau aggregation inhibitor therapy: an exploratory phase 2 study in mild or moderate Alzheimer’s disease. J Alzheimers Dis 44, 705-720

May JM, Qu ZC, Cobb CE. Reduction and uptake of methylene blue by human erythrocytes. American Journal of physiology. 2004. 286(6):C1390-8

Wood C, Nagy H. Methylene blue therapy of viral disease. US20060264423 A1, United States Patent and Trademark Office, 19 May 2006.

Yang ZY, Huang H, Li D et al. Relationship between the ABO Blood Group and the COVID-19 Susceptibility 2020. medRxiv

Yubisui T, Takeshita M, Yoneyama Y. Reduction of methemoglobin through flavin at the physiological concentration by NADPH-flavin reductase of human erythrocytes. J Biochem. 1980. 87(6): 1715-20.

Zhao K, Huang J, Dai D, Feng Y, Liu L & Nie S. Acute myelitis after SARS-CoV-2 infection: a case report. 2020. medRxiv

1. A method of therapeutic treatment of COVID-19 in a subject,
which method comprises administering to said subject a methylthioninium (MT)-containing compound,
wherein said administration provides a total daily oral dose of between 10 and 30 mg of MT to the subject per day, optionally split into 2 or more doses,
or wherein said administration provides a total daily intravenous (IV) dose of between 10 and 25 mg of MT to the subject per day,
wherein the MT-containing compound is an LMTX compound of the following formula: 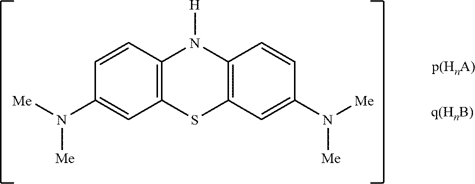
wherein each of HₙA and HₙB (where present) are protic acids which may be the same or different, and wherein p = 1 or 2; q = 0 or 1; n = 1 or 2; (p + q) × n = 2, or a hydrate or solvate thereof.

2. A method as claimed in claim 1 wherein the subject is a human who has been diagnosed with COVID-19, or wherein said method comprises making said diagnosis.

3. A method of prophylactic treatment of COVID-19 in a subject,
which method comprises administering to said subject a methylthioninium (MT)-containing compound,
wherein said administration provides a total daily oral dose of between 10 and 30 mg of MT to the subject per day, optionally split into 2 or more doses,
or wherein said administration provides a total daily intravenous (IV) dose of between 10 and 25 mg of MT to the subject per day,
wherein the MT-containing compound is an LMTX compound of the following formula: 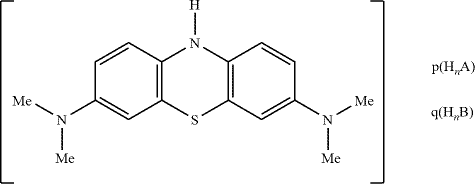
wherein each of HₙA and HₙB (where present) are protic acids which may be the same or different, and wherein p = 1 or 2; q = 0 or 1; n = 1 or 2; (p + q) × n = 2, or a hydrate or solvate thereof.

4. A method as claimed in claim 3 wherein the subject is a human who has been assessed as having suspected or probable COVID-19.

5. A method as claimed in claim 4 wherein the subject is selected from: a subject who has been in close contact with one or more COVID-19 cases; a subject who is at least 65 years old; a subject living in a nursing home, care home, or long-term care facility; a subject with a relevant underlying medical condition.

6. A method as claimed in any one of claims 1 to 5 wherein the total daily dose is between 10 and 25 mg MT (IV) or 12 and 27 mg MT (oral).

7. A method as claimed in claim 6 wherein the total daily dose is between 14 and 20 mg MT.

8. A method as claimed in claim 7 wherein the total daily dose is between 15 and 18 mg MT.

9. A method as claimed in any one of claims 1 to 5 wherein the total daily dose is about 10, 11, 12, 13, 14, 15, 16, 17, 18, 19, 20, 21, 22, 23, 24, 25, 26, 27, 28, 29, or 30 mg MT.

10. A method as claimed in any one of claims 1 to 5 wherein the total daily dose is about 16 mg MT.

11. A method as claimed in any one of claims 1 to 10 wherein the total daily dose of the MT-containing compound is administered as a split oral dose twice a day or three times a day, or is a continuous infusion IV dose, or is an intermittent IV dose which is optionally 2, 4, or 6 times a day.

12. A method as claimed in any one of claims 1 to 11 wherein the treatment is combined with a second compound which is chloroquine or hydroxychloroquine.

13. A method as claimed in any one of claims 1 to 11 wherein the treatment is combined with a second compound or agent selected from: lopinavir-ritonavir; arbidol; azithromycin, remdesivir, favipiravir, actemra; dexamethasone; convalescent plasma.

14. A method as claimed in claim 12 or claim 13 wherein the MT-containing compound and the second compound or agent are administered sequentially within 12 hours of each other.

15. A method as claimed in any one of claims 12 to 14 wherein the subject is pretreated with the second compound or agent prior to commencement of the treatment with the MT-containing compound.

16. A method as claimed in claim 12 or claim 13 wherein the MT-containing compound and the second compound are administered simultaneously, optionally within a single dosage unit.

17. A method as claimed in any one of claims 1 to 16 wherein the MT-containing compound has the following formula, where HA and HB are different mono-protic acids: 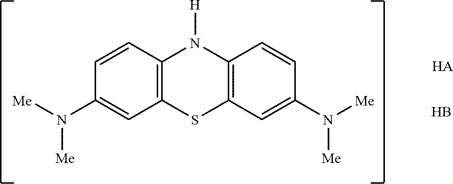
.

18. A method as claimed in any one of claims 1 to 16 wherein the MT-containing compound has the following formula: 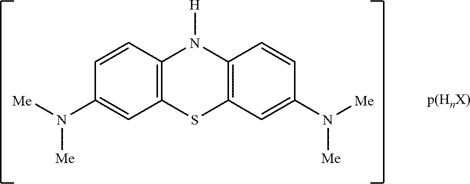
wherein each of HₙX is a protic acid.

19. A method as claimed in any one of claims 1 to 16 wherein the MT-containing compound has the following formula and H₂A is a di-protic acid: 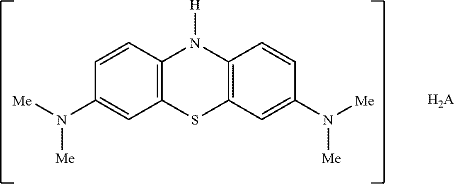
.

20. A method as claimed in claim 18 wherein the MT-containing compound has the following formula and is a bis-monoprotic acid: 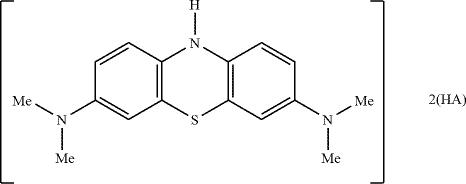
.

21. A method as claimed in any one of claims 1 to 20 wherein the or each protic acid is an inorganic acid.

22. A method as claimed in claim 21 wherein each protic acid is a hydrohalide acid.

23. A method as claimed in claim 21 wherein the or each protic acid is selected from HCl; HBr; HNO_{3;} H₂SO₄.

24. A method as claimed in any one of claims 1 to 20 wherein the or each protic acid is an organic acid.

25. A method as claimed in claim 24 wherein the or each protic acid is selected from H₂CO₃; CH₃COOH; methanesulfonic acid, 1,2-ethanedisulfonic acid, ethansulfonic acid, naphthalenedisulfonic acid, p-toluenesulfonic acid.

26. A method as claimed in any one of claims 1 to 20, or claim 25 wherein the MT-containing compound is LMTM: 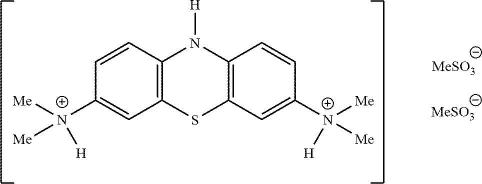
.

27. A method as claimed in claim 26 wherein the total daily dose of LMTM is around 17 mg/day.

28. A method as claimed in claim 27 wherein the dose of LMTM is about 27 mg/once per day.

29. A method as claimed in any one of claims 1 to 20 wherein the MT-containing compound is selected from the list consisting of: 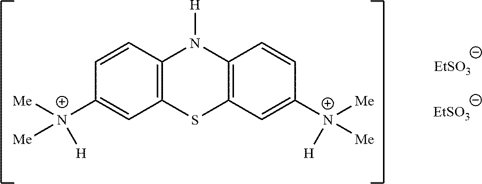 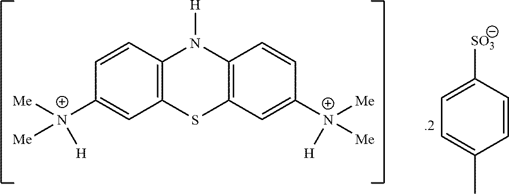 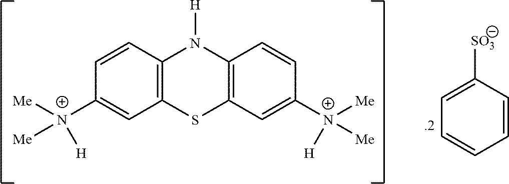 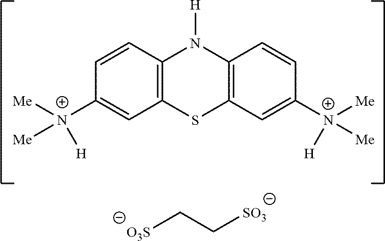 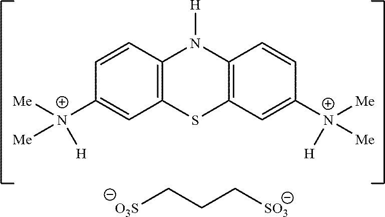 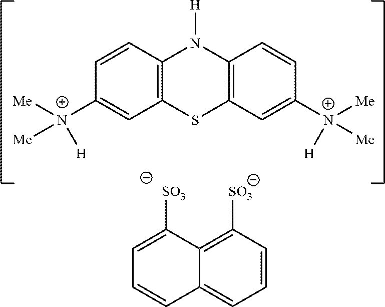
.

30. An MT-containing compound as defined in any one of claims 1 to 29, for use in a method of treatment as defined in any one of claims 1 to 29.

31. Use of an MT-containing compound as defined in any one of claims 1 to 29, in the manufacture of a medicament for use in a method of treatment as defined in any one of claims 1 to 29.
